(19) United States
(12) Patent Application Publication
(10) Pub. No.: US 2016/0310043 A1
(43) Pub. Date: Oct. 27, 2016

(54) Endoscopic Polyp Measurement Tool and Method for Using the Same

(71) Applicant: EndoChoice, Inc., Alpharetta, GA (US)

(72) Inventors: Moshiko Levi, Ganey Tikva (IL); Avi Levy, Herzliya (IL)

(21) Appl. No.: 15/137,573

(22) Filed: Apr. 25, 2016

Related U.S. Application Data
(60) Provisional application No. 62/152,933, filed on Apr. 26, 2015.

(51) Int. Cl. A61B 5/107; A61B 10/04; A61B 1/00

(52) U.S. Cl. CPC A61B 5/1076; A61B 1/00087; A61B 1/00045; A61B 10/04; A61B 5/1079; A61B 2010/045

(57) ABSTRACT

An endoscopy tool for measuring a size of a polyp observed on an internal wall of a body cavity being scanned by an endoscope is provided. The tool is inserted within the body cavity via a service/working channel of the endoscope. The tool has at least a scaled catheter portion having markings for determining a distance of the polyp from a tip of the endoscope; and an operating portion having an elongate member connected to a biopsy tool. The scaled catheter portion has a proximal and a distal end. The elongate member has a distal end and a proximal end coupled with the distal end of the scaled portion. The biopsy tool is connected to the distal end of the elongate member. The determined distance is used for measuring the size of the polyp by an image processing module coupled with the endoscope.

### CROSS-REFERENCE

The present specification relies on U.S. Patent Provisional Application No. 62/152,933, entitled “Endoscopic Polyp Measurement Tool and Methods for Using the Same”, and filed on Apr. 26, 2015, for priority. The above-mentioned application is herein incorporated by reference in its entirety.

### FIELD

The present specification relates generally to endoscopes, and more specifically, to a method for measuring the size of a polyp found in an internal wall of a body cavity during an endoscopic examination.

### BACKGROUND

An endoscope is a medical instrument used for examining and treating internal body cavities such as the alimentary canals, airways, the gastrointestinal system, and other organ systems. Conventional endoscopes are usually an elongated tubular shaft, rigid or flexible, having a video camera and a fiber optic light guide for directing light from an external light source situated at a proximal end of the tube to a distal tip. Also, most endoscopes are provided with one or more channels, through which medical devices, such as forceps, probes, and other tools, may be passed. Further, during an endoscopic procedure, fluids, such as water, saline, drugs, contrast material, dyes, or emulsifiers are often introduced or evacuated via the shaft. A plurality of channels, one each for introduction and suctioning of liquids, may be provided within the shaft.

Endoscopes have attained great acceptance within the medical community, since they provide a means for performing procedures with minimal patient trauma, while enabling the physician to view the internal anatomy of the patient. Over the years, numerous endoscopes have been developed and categorized according to specific applications, such as cystoscopy, colonoscopy, laparoscopy, upper gastrointestinal (GI) endoscopy among others. Endoscopes may be inserted into the body's natural orifices or through an incision in the skin.

Some endoscopes have a front camera as well as one or more side cameras for viewing an internal organ, such as the colon, and an illuminator for illuminating the field of view of the camera(s). The camera(s) and illuminators are located in a tip of the endoscope and are used to capture images of the internal walls of the body cavity being endoscopically scanned. The captured images are sent to a control unit coupled with the endoscope via one of the channels present in the scope shaft, for being displayed on a screen coupled with the control unit.

While endoscopes help in detection and treatment of a number of diseases in a relatively non-invasive manner, endoscopes suffer from the drawback of having a limited field of view. The field of view is limited by the narrow internal geometry of organs as well as the insertion port, which may be body's natural orifices or an incision in the skin. Further, in order to determine the exact position/orientation of an endoscope tip within a body cavity, a physician usually has to rely on experience and intuition.

Sometimes, irregularities such as polyps are observed as formations on internal walls of a body cavity being scanned. Accurate measurement of the polyp size during an endoscopic procedure such as colonoscopy, gastroscopy, bronchoscopy and the like is important because there may be a direct correlation of polyp size with colon cancer or other diseases. Major studies of colorectal neoplasms have measured polyp size differently and inconsistently. It is also well documented that endoscopists frequently underestimate polyp size.

For the early detection and cure of many diseases such as cancer, it is essential that the polyp size be determined accurately. Hence, there is need for a tool and a method for using such tool that enables an operating physician to assess the size of an observed polyp with increased accuracy during an endoscopic procedure.

### SUMMARY

In some embodiments, the present specification discloses an endoscopy tool for measuring a size of a polyp observed on an internal wall of a body cavity being scanned by an endoscope, wherein the endoscopy tool is adapted to be inserted within the body cavity via a channel of the endoscope, the endoscopy tool comprising: a scaled catheter portion having a plurality of markings for determining a unit of distance within the body cavity, wherein the scaled catheter portion has a proximal and a distal end; and an operating portion comprising an elongate member coupled with a biopsy tool, wherein the elongate member has a distal end and a proximal end coupled with the distal end of the scaled portion and wherein the biopsy tool is coupled with the distal end of the elongate member; and a processing unit adapted to determine the unit of distance from said plurality of markings using a scale conversion and to transmit data to a display, wherein said data is indicative of the unit of distance.

Optionally, the biopsy tool comprises at least one of a pair of jaws, a needle, and a net.

Optionally, a distance between any two markings on the scaled catheter portion ranges from one of 0.01 mm to 1 mm.

Optionally, a distance between markings closer to the distal end is smaller as compared to a distance between markings on the scaled catheter portion farther away from the distal end.

Optionally, the elongate member comprises a plurality of markings for determining a distance of the polyp from the distal end of the scaled catheter portion. Optionally, lengths of the elongate portion and the biopsy tools are pre-defined and wherein the lengths are adapted to be used for determining the distance of the polyp from the tip of the endoscope. Optionally, the endoscopy tool has a length ranging from between 210 cm to 240 cm.

Optionally, the endoscopy tool is adapted to exit the endoscope via an opening in a tip portion of the endoscope.

Optionally, the biopsy tool comprises a jaw set having a plurality of flanges, a base end of each of the flanges of the jaw set being coupled with the distal end of the elongate member, the distal end of the elongate member comprising a needle having a tip extending from the distal end of the elongate member in the plane of the elongate member, the tip of the needle being positioned in close proximity of the polyp for determining the distance of the polyp from the tip of the endoscope, wherein the flanges of the jaw set may be placed in a first closed position and a second open position by operating a control; the flanges lying in a plane parallel to the plane of the elongate member in the first position enclosing the needle tip; and the flanges lying in a plane perpendicular to the plane of the elongate member in the second position exposing the needle tip.

Optionally, the processing unit is configured to determine a size of the polyp by using the unit of distance from said plurality of markings and a distance between two edges of the polyp.

In some embodiments, the present specification discloses a method of measuring a size of a polyp observed on an internal wall of a body cavity being scanned by an endoscope by using an endoscopy tool comprising at least a scaled catheter portion having a plurality of markings for determining a unit of distance, the scaled catheter portion having a proximal and a distal end; and an operating portion comprising an elongate member coupled with a biopsy tool, the elongate member having a distal end and a proximal end coupled with the distal end of the scaled portion, the biopsy tool being coupled with the distal end of the elongate member; the method comprising the steps of: examining internal walls of the body cavity via a displayed endoscopic image; locating a polyp on an internal wall of the body cavity via the displayed endoscopic image; marking the location of the polyp on the displayed endoscopic image; inserting the endoscopy tool into the body cavity via a service channel of the endoscope; placing a distal tip of the biopsy tool in proximity to the polyp location, the scaled catheter portion of the tool extending from an opening in the tip of the endoscope; activating a scale function by operating a control provided on the endoscope for determining a size of the polyp by determining a distance of the polyp from a tip of the endoscope and a predefined algorithm; and displaying the determined size of the polyp in the endoscopic image.

Optionally, the distance of the polyp from the tip of the endoscope is determined by using the plurality of markings of the scaled catheter portion visible in the endoscopic image.

Optionally, the predefined algorithm is a size determination algorithm for measuring the size of the polyp by using the distance of the polyp from the tip of the endoscope.

Optionally, the markings on the scaled catheter portion represent a predefined unit of measurement.

In some embodiments, the present specification discloses a method of measuring a size of a polyp observed on an internal wall of a body cavity being scanned by an endoscope by using an endoscopy tool comprising at least a scaled catheter portion having a plurality of markings for determining a distance of the polyp from a tip of the endoscope, the scaled catheter portion having a proximal and a distal end; and an operating portion comprising an elongate member coupled with a biopsy tool, the elongate member having a distal end and a proximal end coupled with the distal end of the scaled portion, the biopsy tool being coupled with the distal end of the elongate member; the method comprising the steps of: examining internal walls of the body cavity via a displayed endoscopic image; locating a polyp on an internal wall of the body cavity via the displayed endoscopic image; marking the location of the polyp on the displayed endoscopic image; inserting the endoscopy tool into the body cavity via a service channel of the endoscope; placing a distal tip of the endoscopy tool in closed proximity of the polyp location, the scaled catheter portion of the tool extending from a tip of the endoscope; activating a scale function by operating a control provided on the endoscope for marking a first and a second opposing edge of the polyp by a marker displayed in the endoscopic image; determining the size of the polyp by using the number of markings of the scaled catheter portion visible in the endoscopic image, the distance between the two marked edges of the polyp and a predefined size determination algorithm; and displaying the determined size of the polyp in the endoscopic image.

Optionally, the biopsy tool is positioned in close proximity of the polyp for determining the distance of the polyp from the tip of the endoscope.

Optionally, the markings on the scaled catheter portion represent a predefined unit of measurement.

Optionally, the method further comprises rendering a three dimensional image of the polyp by measuring a longitudinal dimension of the polyp and a height of the polyp.

Optionally, the longitudinal dimension and the height of the polyp is measured by placing a distal end of the endoscopy medical tool on a proximal end of the polyp, a distal end of the polyp and across the length of the polyp and using the predefined size determination algorithm.

The present specification discloses an endoscopy tool for measuring a size of a polyp observed on an internal wall of a body cavity being scanned by an endoscope, the tool being inserted within the body cavity via a channel of the endoscope, the tool comprising: at least a scaled catheter portion having a plurality of markings for determining a distance of the polyp from a tip of the endoscope, the scaled catheter portion having a proximal and a distal end; and an operating portion comprising an elongate member coupled with a biopsy tool, the elongate member having a distal end and a proximal end coupled with the distal end of the scaled portion, the biopsy tool being coupled with the distal end of the elongate member; wherein the determined distance is used for measuring the size of the polyp by an image processing module coupled with the endoscope.

In some embodiments, the present specification discloses a method of measuring a size of a polyp observed on an internal wall of a body cavity being scanned by an endoscope by using an endoscopy tool comprising at least a scaled catheter portion having a plurality of markings for determining a distance of the polyp from a tip of the endoscope, the scaled catheter portion having a proximal and a distal end; and an operating portion comprising an elongate member coupled with a biopsy tool, the elongate member having a distal end and a proximal end coupled with the distal end of the scaled portion, the biopsy tool being coupled with the distal end of the elongate member; the method comprising the steps of: examining internal walls of the body cavity via a displayed endoscopic image; locating a polyp on an internal wall of the body cavity via the displayed endoscopic image; marking the location of the polyp on the displayed endoscopic image; inserting the endoscopy tool into the body cavity via a service channel of the endoscope; placing a distal tip of the endoscopy tool in closed proximity of the polyp location, the scaled catheter portion of the tool extending from a tip of the endoscope; activating a scale function by operating a control provided on the endoscope for determining the size of the polyp by using the plurality of markings of the scaled catheter portion visible in the endoscopic image and a predefined size determination algorithm; and displaying the determined size of the polyp in the endoscopic image.

In some embodiments, the present specification is directed toward a method of measuring a size of a polyp observed on an internal wall of a body cavity being scanned by an endoscope by using an endoscopy tool comprising at least a scaled catheter portion having a plurality of markings for determining a distance of the polyp from a tip of the endoscope, the scaled catheter portion having a proximal and a distal end; and an operating portion comprising an elongate member coupled with a biopsy tool, the elongate member having a distal end and a proximal end coupled with the distal end of the scaled portion, the biopsy tool being coupled with the distal end of the elongate member; the method comprising the steps of: examining internal walls of the body cavity via a displayed endoscopic image; locating a polyp on an internal wall of the body cavity via the displayed endoscopic image; marking the location of the polyp on the displayed endoscopic image; inserting the endoscopy tool into the body cavity via a service channel of the endoscope; placing a distal tip of the endoscopy tool in closed proximity of the polyp location, the scaled catheter portion of the tool extending from a tip of the endoscope; activating a scale function by operating a control provided on the endoscope for marking a first and a second opposing edge of the polyp by a marker displayed in the endoscopic image; determining the size of the polyp by using the number of markings of the scaled catheter portion visible in the endoscopic image, the distance between the two marked edges of the polyp and a predefined size determination algorithm; and displaying the determined size of the polyp in the endoscopic image.

The aforementioned and other embodiments of the present shall be described in greater depth in the drawings and detailed description provided below.

### BRIEF DESCRIPTION OF THE DRAWINGS

These and other features and advantages of the present invention will be appreciated, as they become better understood by reference to the following detailed description when considered in connection with the accompanying drawings, wherein:

FIG. 1 illustrates a multiple viewing elements endoscopy system in which a tool and method for determining the size of a polyp may be implemented;

FIG. 2A illustrates another view of an endoscopy system in which a method for determining the size of a polyp may be implemented;

FIG. 2B is a block diagram illustrating the endoscopy system in which the method for determining the size of a polyp provided by the present specification may be implemented;

FIG. 3A illustrates one view of an endoscopy tool comprising a forceps portion, in accordance with an embodiment of the present specification;

FIG. 3B illustrates another view of the endoscopy tool comprising a forceps portion, in accordance with an embodiment of the present specification;

FIG. 4 illustrates an endoscopy tool comprising a snare portion, in accordance with another embodiment of the present specification;

FIG. 5A illustrates an internal view of a body cavity being scanned by an endoscope;

FIG. 5B illustrates a location of a polyp marked on an internal wall of the body cavity being scanned by an endoscope, shown in FIG. 4;

FIG. 5C illustrates an endoscopy tool for measuring a polyp when extended into a body cavity, in accordance with an embodiment of the present specification;

FIG. 5D illustrates an endoscopy tool extending and deployed from an endoscope's tip section, in accordance with an embodiment of the present specification;

FIG. 5E illustrates an exemplary handle of an endoscope comprising a control for operating a ‘scale’ function, in accordance with an embodiment of the present specification;

FIG. 5F illustrates an image of the internal walls of a body cavity displaying a polyp location as well as the size of the polyp, in accordance with an embodiment of the present specification;

FIG. 5G illustrates an image of the internal walls of a body cavity showing an on-screen marker and an endoscopy tool tip placed in proximity to a polyp location, in accordance with an embodiment of the present specification;

FIG. 5H illustrates an image of the internal walls of a body cavity showing a marker marking a first edge of a polyp, in accordance with an embodiment of the present specification;

FIG. 5I illustrates an image of the internal walls of a body cavity showing a marker marking a second edge of the polyp, in accordance with an embodiment of the present specification;

FIG. 5J illustrates an image of the internal walls of a body cavity displayed on a monitor screen with a size of an observed polyp displayed therein, in accordance with an embodiment of the present specification;

FIG. 5K illustrates an image of the internal walls of a body cavity displayed on a monitor screen with a size of an observed polyp displayed therein, in accordance with an embodiment of the present specification;

FIG. 6 is a flowchart illustrating the steps for determining the size of a polyp observed within a body cavity being scanned by an endoscope, in accordance with an embodiment of the present specification; and

FIG. 7 is a flowchart illustrating the steps for determining the size of a polyp observed within a body cavity being scanned by an endoscope, in accordance with another embodiment of the present specification.

### DETAILED DESCRIPTION

The present specification provides a device and method for determining the size of a polyp located along an internal wall of a body cavity, as observed via an endoscope. The present specification provides an endoscopy tool comprising at least a scaled portion ending in forceps, snares or other medical tools having a distal tip. Once a polyp is endoscopically observed on a monitoring screen, the endoscopy tool is inserted through an insertion/working channel of the endoscope and drawn out into the observed lumen such that the tip of the corresponding forceps, snare, or other device is in close proximity to the polyp. In an embodiment, the scaled markings visible on the portion of the tool extending beyond the endoscope tip enables calculation of a distance of the endoscope tip from the polyp. In another embodiment, the tip of the corresponding forceps, snare, or other medical device is first used to mark a first proximal end of the polyp on a display screen and then used to mark a second, opposite distal end of the polyp, thereby enabling calculation of a size of the polyp.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

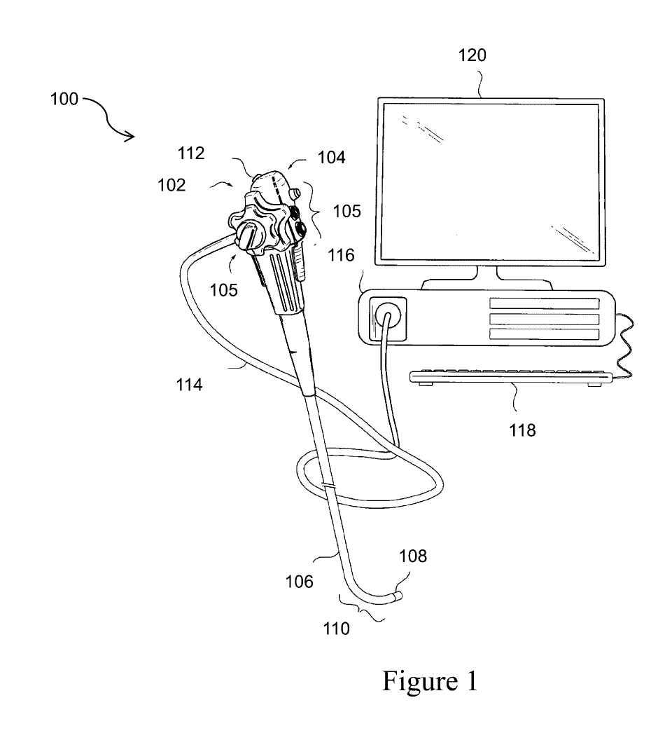

FIG. 1 illustrates a multiple viewing elements endoscopy system in which the method for determining the size of a polyp may be implemented. As would be apparent to persons of skill in the art, the method for determining the size of a polyp by using a tool having scaled markings described in the present specification may be implemented in any endoscope comprising one or more viewing elements for capturing images of the insides of a body cavity.

Reference is now made to FIG. 1 which shows a semi-pictorial view of a multiple viewing elements element endoscopy system 100. System 100 may include a multiple viewing elements endoscope 102. Multiple viewing elements endoscope 102 may include a handle 104, from which an elongated shaft 106 emerges. Elongated shaft 106 terminates with a tip section 108 which is turnable by way of a bending section 110. Handle 104 may be used for maneuvering elongated shaft 106 within a body cavity; the handle may include one or more knobs and/or buttons/switches 105 which control bending section 110 as well as functions such as fluid injection and suction and toggling between the multiple viewing elements of tip section 108. Handle 104 further includes a service/working channel opening 112 through which surgical tools may be inserted.

Tip section 108 may include multiple optical assemblies, which comprise viewing elements. In accordance with an embodiment, tip section 108 includes a front optical assembly and one or two side optical assemblies. In another embodiment, tip 108 may include only a front optical assembly.

In addition, tip section 108 may include at least one service/working channel exit point. In accordance with an embodiment, tip section 108 includes a front service/working channel exit point and at least one side service channel exit point. In another embodiment, tip section 108 may include two front service/working channel exit points.

A utility cable 114 may connect between handle 104 and a controller 116. Utility cable 114 may include therein one or more fluid channels and one or more electrical channels. The electrical channel(s) may include at least one data cable for receiving video signals from the front and side-viewing elements, as well as at least one power cable for providing electrical power to the viewing elements and to at least one discrete illuminator.

Controller 116 may govern power transmission to the tip section 108 of endoscope 102, such as for the tip section's viewing elements and illuminators. Controller 116 may further control one or more fluid, liquid and/or suction pump which supply corresponding functionalities to endoscope 102. One or more input devices, such as a keyboard 118, a computer, a touch screen, a voice controller and the like, may be connected to controller 116 for the purpose of human interaction with the controller. In another configuration, an input device, such as a keyboard, or a touch screen, may be integrated with the controller in a same casing.

A display 120 may be connected to controller 116, and configured to display images and/or video streams received from the viewing elements of the endoscope 102. Display 120 may further be operative to display a user interface for allowing a human operator to set various features of system 100. Display 120 may further be a multi-monitor display that is either discrete or contiguous.

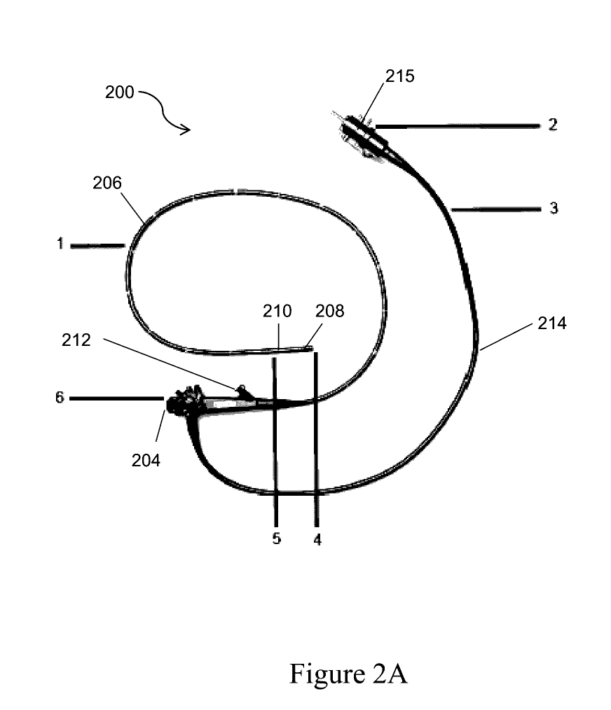

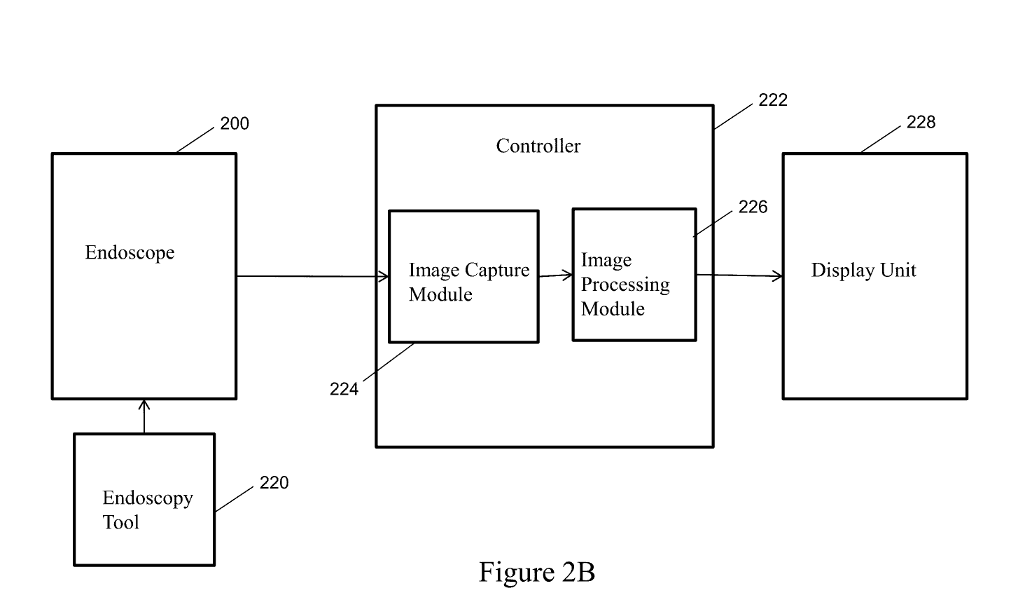

FIG. 2A illustrates another view of an endoscope in which the method for determining the size of a polyp provided by the present specification may be implemented. Endoscope 200 comprises a handle 204, from which an elongated shaft 206 emerges. Elongated shaft 206 terminates with a tip section 208 which is turnable by way of a bending section 210. Handle 204 may be used for maneuvering elongated shaft 206 within a body cavity. In various embodiments, handle 204 comprises a service channel opening 212 through which an endoscopy tool may be inserted. In various embodiments, handle 204 comprises at least one service channel opening (not shown) through which an endoscopy tool may be inserted.

Further in various embodiments, tip 208 includes at least one service/working channel exit point to provide an exit for the endoscopy tool. In one embodiment, the endoscopy tool is placed in close proximity to a polyp on a wall of the body cavity being endoscopically examined. A utility cable 214 is used to connect handle 204 with a controller via an electrical connector 215. The controller governs power transmission to the tip section 208. In one embodiment, the controller also controls one or more fluid, liquid and/or suction pump which supply corresponding functionalities to endoscope 200. One or more input devices, such as a keyboard, a computer, a touch screen, a voice controller and the like, may be connected to the controller to enable human interaction with the controller. The controller comprises an image processing module which enables determination of the polyp size measured by using the endoscopy tool of the present specification. A display may be connected to the controller, and configured to display images and/or video streams received from the viewing elements of the endoscope 200.

FIG. 2B is a block diagram illustrating the endoscopy system in which the method for determining the size of a polyp provided by the present specification may be implemented. Referring to both FIGS. 2A and 2B, endoscope 200 comprises a handle 204 having a service channel opening 212 through which endoscopy tool 220 may be inserted. Further, tip 208 of endoscope 200 includes at least one service/working channel exit point from which the endoscopy tool 220 exits for being placed in close proximity to a polyp on a wall of the body cavity being endoscopically examined. A utility cable 214 is used to connect handle 204 with controller 222 via an electrical connector 215. Controller 222 comprises an image capturing module 224 for capturing the image of the polyp obtained via one or more viewing elements of the endoscope 200, and an image processing module 226 which enables determination of the polyp size measured by using the endoscopy tool 220 and a predefined size determination algorithm. A display unit 228 connected to the controller is configured to display images and/or video streams received from the viewing elements of the endoscope 200 along with the measured size of the polyp.

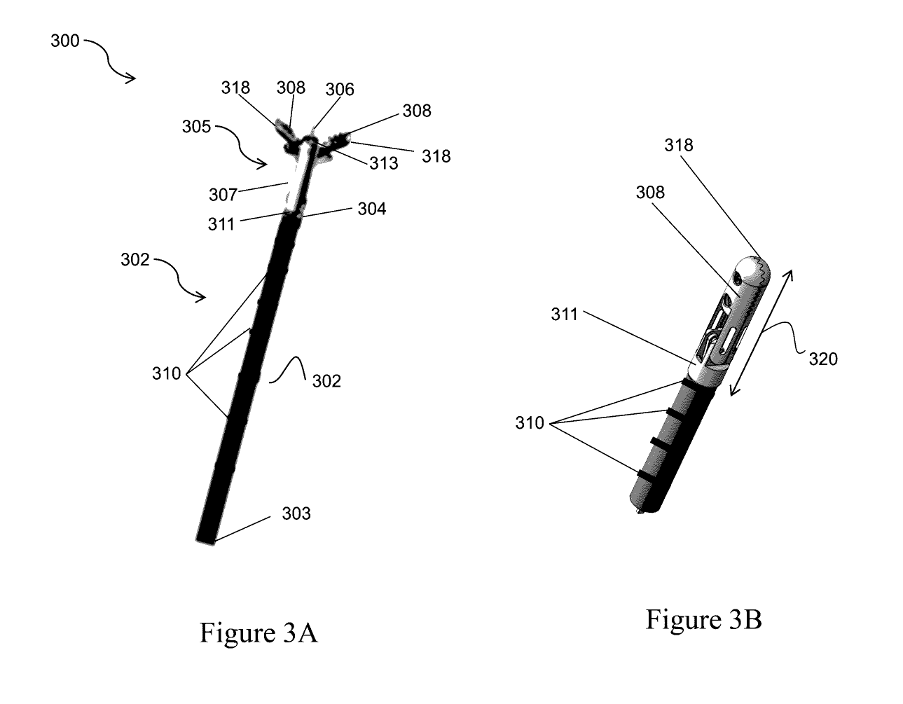

FIG. 3A is an illustration of an exemplary endoscopy tool, in accordance with an embodiment of the present specification. Endoscopy tool 300 comprises a scaled catheter portion 302 having a proximal end 303 and a distal end 304; and a forceps portion 305 comprising an elongate portion 307 and a jaw set 308. Forceps portion 305 is positioned at the distal end 304 of the endoscopy tool 300. Elongate portion 307 has a proximal end 311 which is coupled with the distal end 304 of scaled catheter portion 302. A forceps needle having a needle tip 306 protrudes from a distal end 313 of the elongate portion 307. FIG. 3A illustrates a first position of jaw set 308, wherein in the first position, the jaw set 308 is open. FIG. 3B illustrates another view of the endoscopy tool, in accordance with an embodiment of the present specification, wherein the jaw set 308 is in a second position, and wherein in the second position, the jaw set 308 is closed. In the second position shown in FIG. 3B distal ends 318 of the jaw set 308 close around the forceps needle tip 306 covering the needle tip 306 completely, thereby protecting the needle tip 306 as well as walls of the endoscopic channel through which the endoscopy tool 300 may be inserted for measuring a polyp size.

In an embodiment, catheter portion 302 is elongate and comprises markings 310 denoting a predefined scale. For example, in an embodiment the distance between two adjacent markings 310 may range from 0.01 mm to 1.0 mm. In another embodiment, the distance between two adjacent markings 310 may range from 0.5 mm to 1.0 mm. In another embodiment, the distance between two adjacent markings 310 may range from 0.5 mm to 5.0 mm. In an embodiment, the distances between markings closer to the distal end 304 are shorter as compared to the distances between markings on the scaled portion 302 farther away from the distal end 304. This provides for smaller scale measurements close to the pointed needle tip 306 and distal end 318.

In an embodiment, length 320 of the forceps portion 305 may be scaled and marked in a similar manner as the catheter portion 302. In an embodiment, the length 320 of the forceps portion 305 is pre-defined and input into the controller (116, shown in FIG. 1) of the endoscope. The controller 116 enables determination of the distance of a polyp viewed from a front or a side viewing element of the endoscope when the endoscopy tool 300 is inserted into a body cavity, by using the pre-defined length 320. In an embodiment, a distance between the distal end 318 and the forceps needle tip 306 may be also be pre-defined.

In various embodiments, other medical tools such as but not limited to snares may be coupled with the scaled catheter portion 302 of the endoscopy tool for measuring polyp sizes. The scaled catheter portion 302 may comprise a female or male mating interface that can be removably coupled to a complimentary mating interface on the non-working end of the tool head, such as a snare, a needle, a forceps, or a forceps/needle combination. In this manner, a single scaled catheter portion may be used with a plurality of different tools.

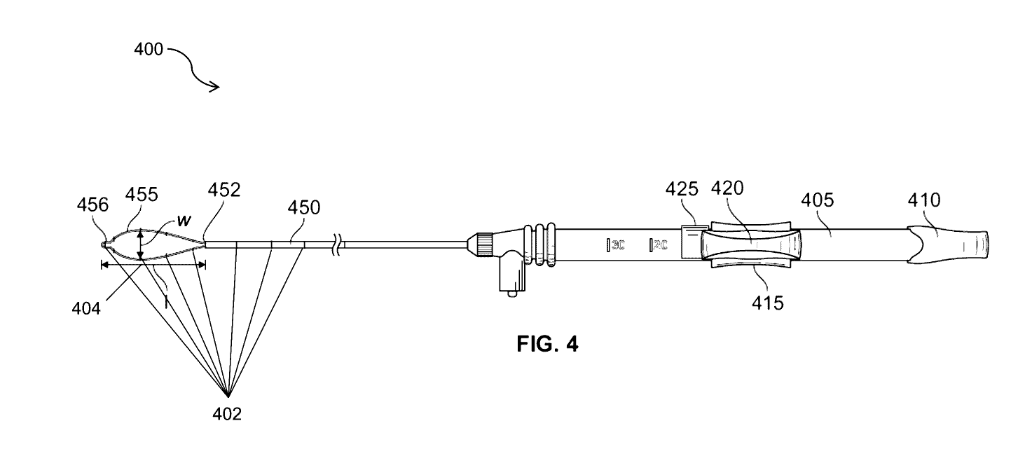

FIG. 4 illustrates another endoscopy tool comprising a snare for measuring a polyp size, in accordance with an embodiment of the present specification. U.S. patent application Ser. No. 14/162,691, which relates to the Application of the present specification, entitled “Surgical Snare Device” and filed on Jan. 23, 2014 is one example of an endoscopy tool and is herein incorporated by reference in its entirety. In addition, U.S. patent application Ser. No. 14/834,232, which relates to the Application of the present specification, entitled “Surgical Snare Device” and filed on Aug. 24, 2015 is another example of an endoscopy tool and is herein incorporated by reference in its entirety. Both of them may be fixably or removably combined with a scaled catheter portion 302.

The snare device 400 shown in FIG. 4 includes a handle and an elongated tubular sheath 450 attached to the distal end of the handle. The handle comprises a first handle assembly 405, a second handle assembly 415, a cautery tip housing, and a collar 425. The first handle assembly 405 includes a cylindrical body with a proximal end and a distal end, a finger ring 420 at the proximal end, and a channel extending from a point proximate its proximal end to a point proximate its distal end. The cautery tip housing is attached to and rotatable coaxially about the distal end of the first handle assembly. The second handle assembly 415 includes a center body portion having a proximal end and a distal end with a finger ring 420 positioned on both sides. The second handle assembly 415 and collar 425 are coupled to, and slidably movable along via the channel of, the first handle assembly 405. The collar 425 includes a proximal end and a distal end and is positioned distal to the second handle assembly 415. A rigid driveshaft having a proximal end and a distal end is positioned within the channel and has its proximal end attached to the distal end of the center body of the second handle assembly 415. The driveshaft extends distally through the channel, into the cautery tip housing, wherein it is in electrical contact with the cautery tip, and is attached at its distal end to the proximal end of a drive wire. An elongate tubular sheath 450 is attached to the distal end of the first handle assembly 405 and extends distally from the handle. A flexible drive wire extends distally within the tubular sheath 450 and has a snare loop 455 attached at its distal end. The proximal end of the flexible drive wire is permanently fixed to the rigid driveshaft. The snare loop 455 rests within the sheath in a collapsed configuration and is deployable, by sliding the second handle assembly 415 distally along the first handle assembly 405, to an expanded configuration out of the distal end of the sheath 450.

Advancing the second handle assembly 415 to the position depicted in FIG. 4 deploys the snare loop 455 from the sheath 450 such that the snare loop 455 has an internal diameter or width w of precisely 10 mm, corresponding to the marking on the first handle assembly 405. In addition, when the second handle assembly is at the position depicted in FIG. 4, the snare loop 455 has a length, not including the v-shaped notch 456 discussed below, of 25 mm from the opening 452 at the distal end of the sheath 450 to the distal end of the snare loop 455.

Also visible in FIG. 4 is the v-shaped notch 456 at the distal end of the snare loop. The notch 456 helps the snare loop 455 keep its teardrop shape as it is extended from and retracted into the sheath 450. To deploy the snare loop 455, a user moves the second handle assembly 415 by holding the first handle assembly in place with finger ring 410 and simultaneously using finger rings 420 to slide the second handle assembly 415 distally along the first handle assembly 405. When the snare loop 455 is in the withdrawn position within the sheath 450, the wires of the two ends of the snare loop are positioned parallel to one another. As the snare loop 455 is deployed, the wires twist over each other and then eventually untwist once the snare loop 455 has been deployed to the desired width ‘w’.

In an embodiment, length 404 of the snare loop 455 and a pre-determined portion of the sheath 450 may be scaled and marked as 402, in a similar manner as the catheter portion 302 shown in FIGS. 3A and 3B. In an embodiment, the length of the marked portion 402 is pre-defined and fed into the controller (116, shown in FIG. 1) of the endoscope. The controller 116 enables determination of the distance of a polyp viewed from a front or a side viewing element of the endoscope when the endoscopy tool 400 is inserted into a body cavity, by using the pre-defined length 402.

In an embodiment, the length of the endoscopy tool of the present specification ranges between 210 to 240 centimeters. In an embodiment, the endoscopy tool is made of any suitable material being used to make surgical tools.

In an embodiment, once a polyp is observed within a body cavity being endoscopically scanned, the endoscopy tool is inserted within the body cavity via a service channel of the endoscope. The polyp as well as the endoscopy tool is observed on a monitoring screen coupled with the endoscope. Once a tip of the tool is in close proximity of the polyp, an image processor coupled with the endoscope enables determination of the size of the polyp by using the number of markings of the tool visible on the screen and a predefined algorithm.

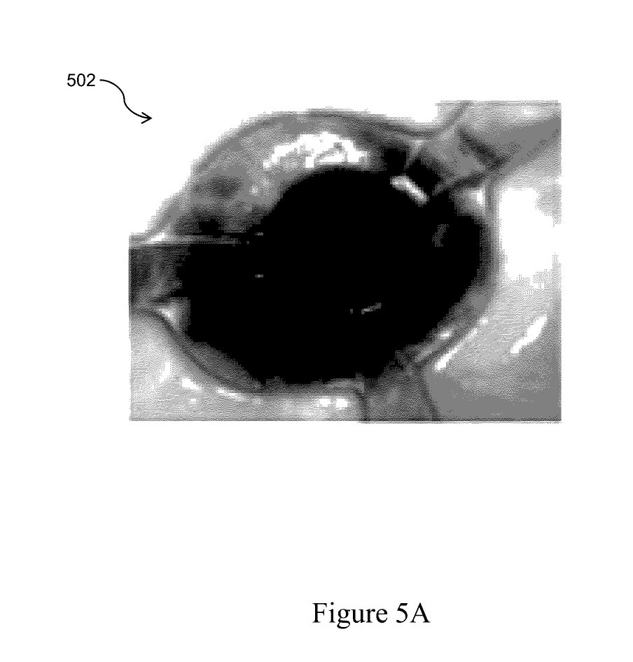

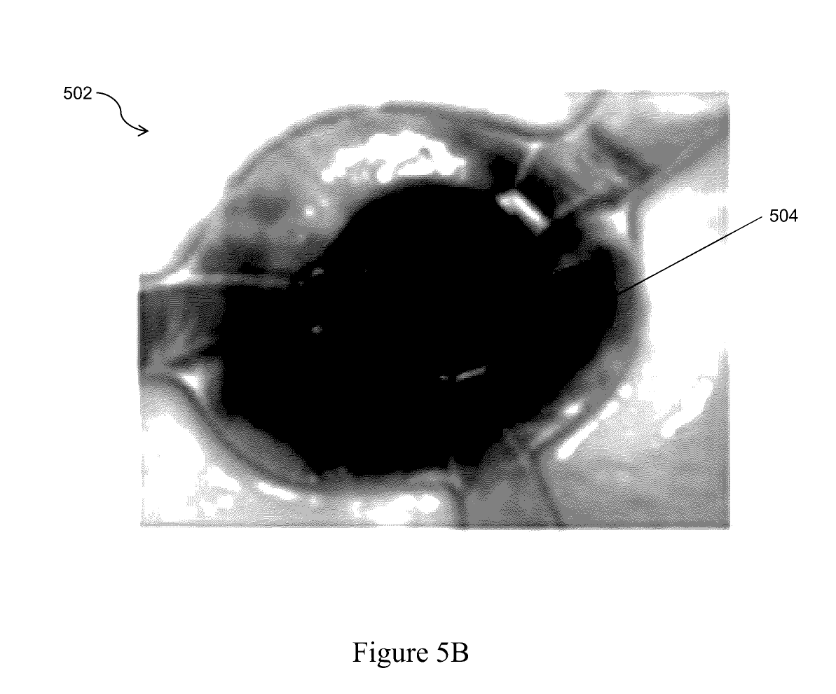

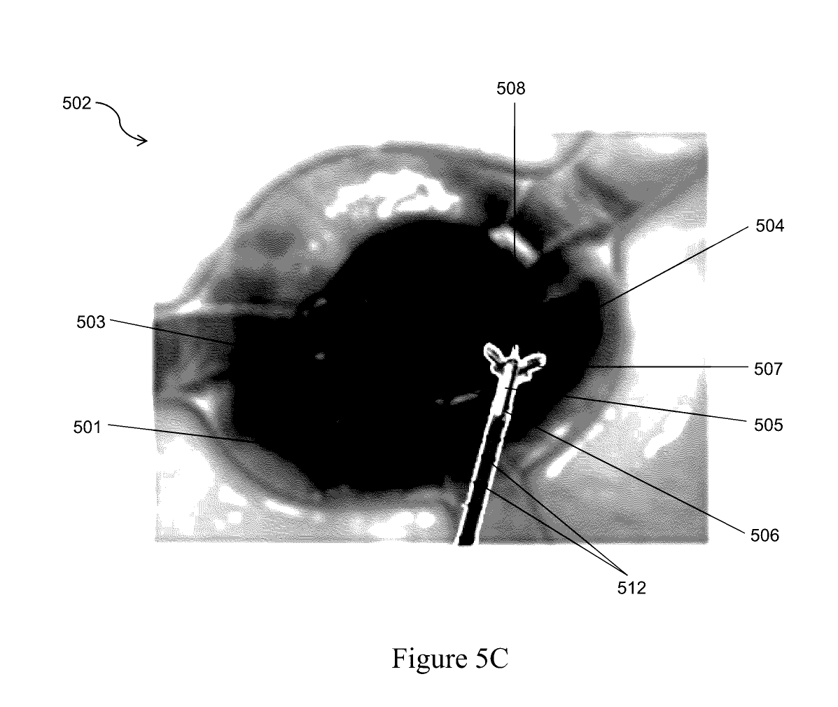

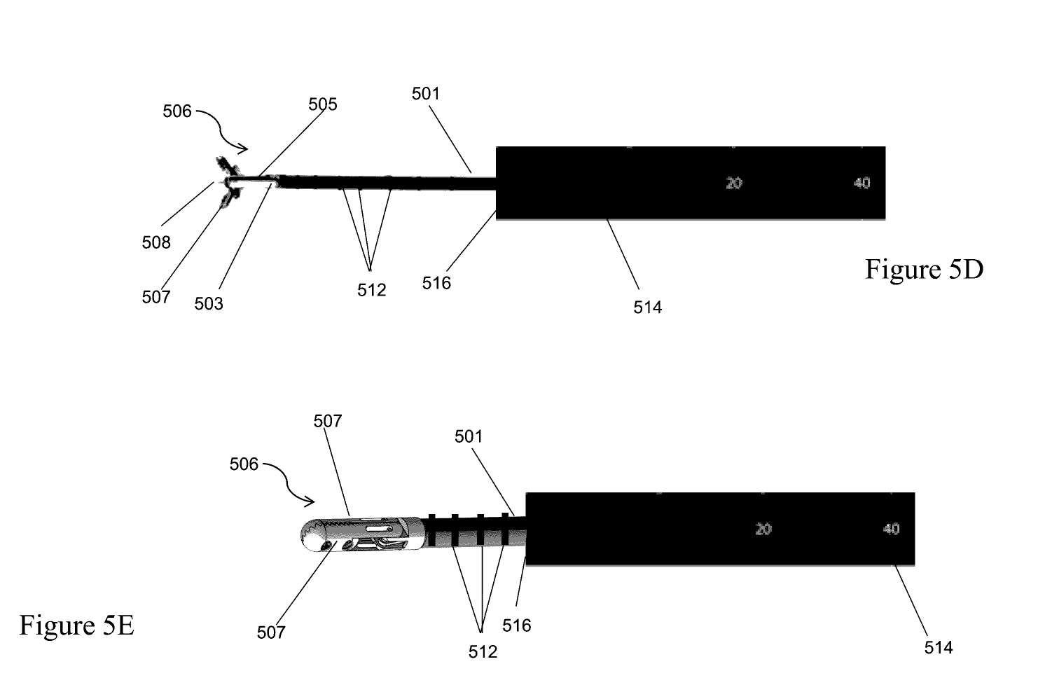

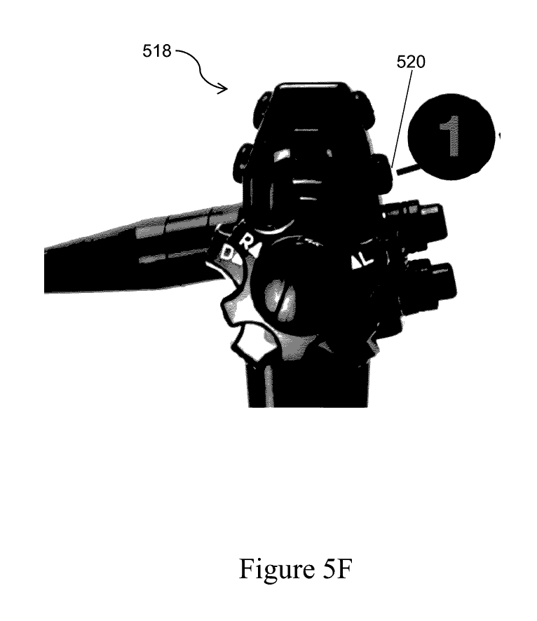

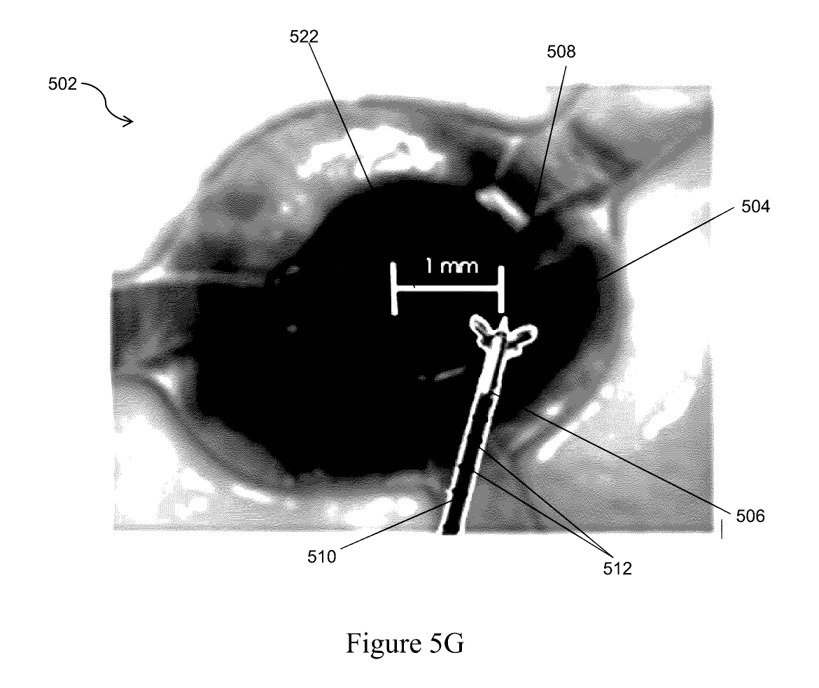

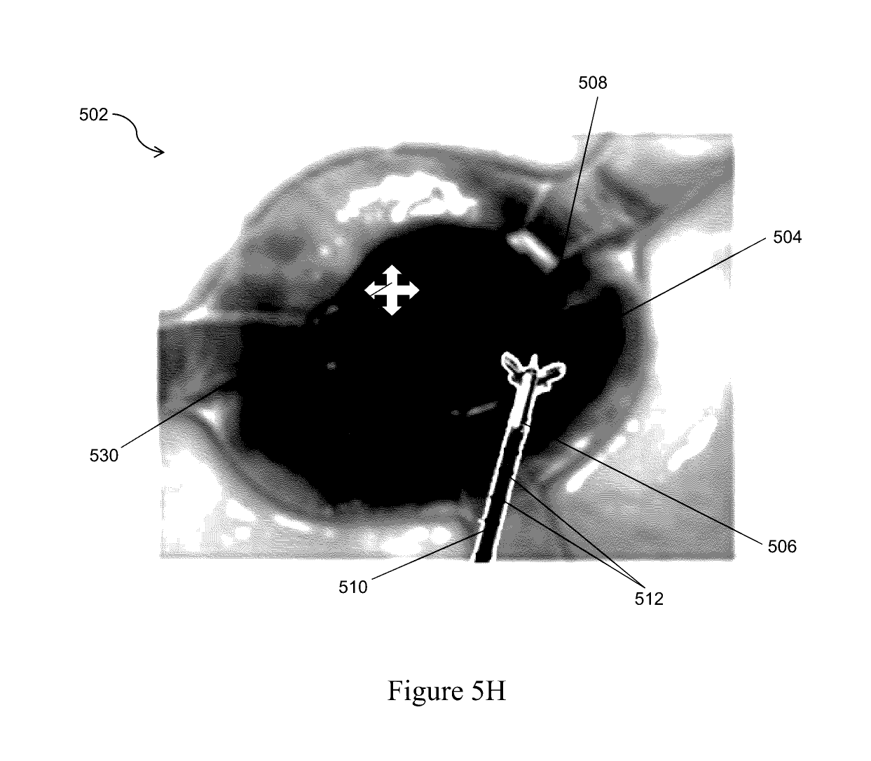

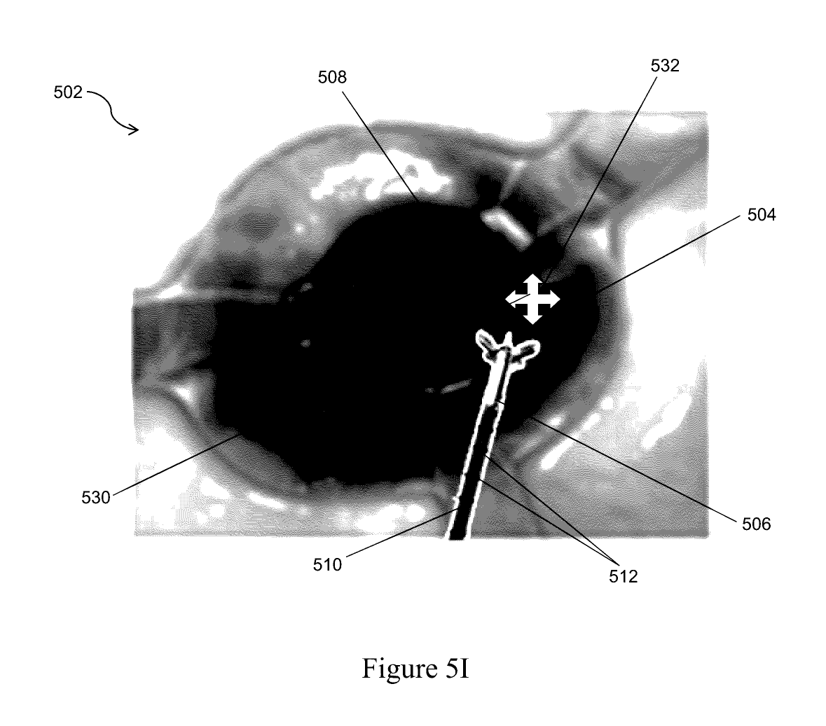

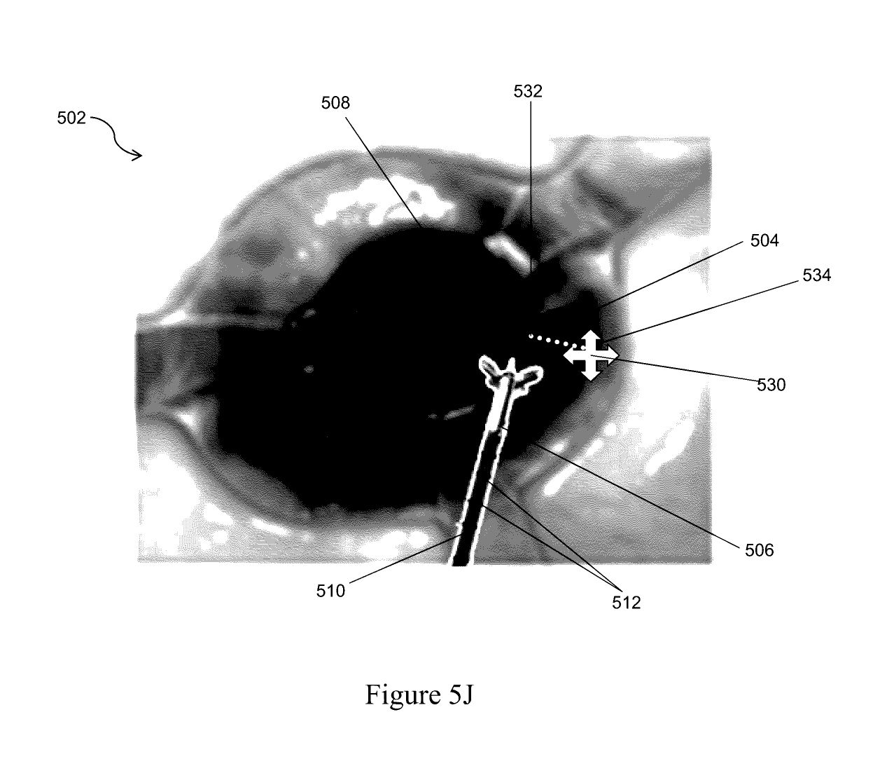

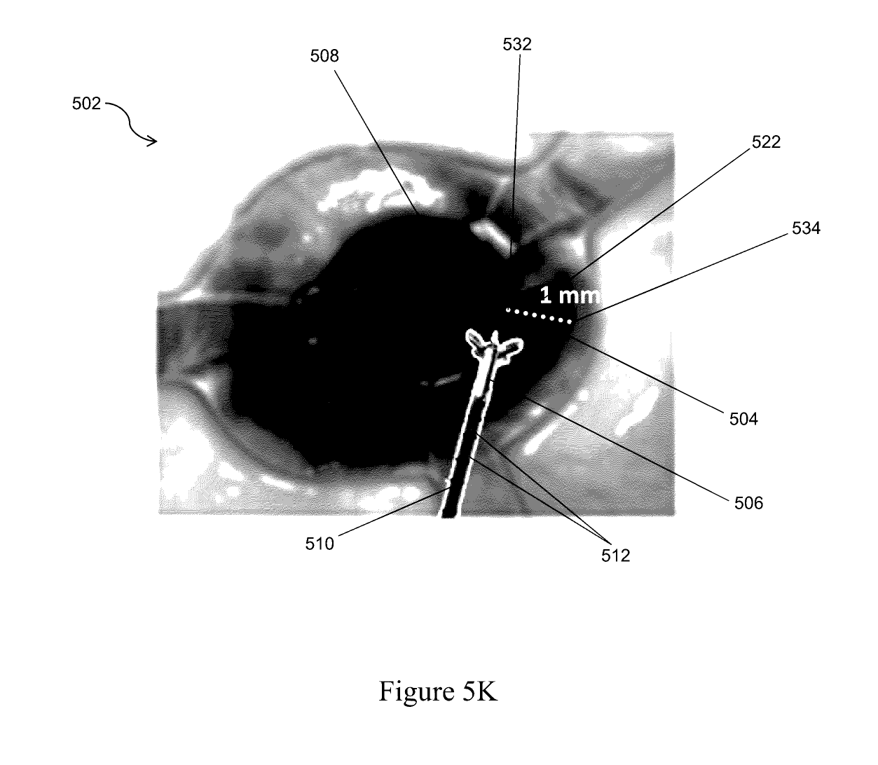

FIG. 5A illustrates an internal view of a body cavity, as viewed from a viewing element located on a distal tip (108, FIG. 1) of an endoscope. In an embodiment, FIG. 5A illustrates an image of the internal walls 502 of a human colon as may be seen on a monitor screen coupled with the viewing element of the endoscope scanning the colon. FIG. 5B illustrates the location of a polyp marked on an internal wall of a body cavity being scanned by the endoscope. In an embodiment, when a polyp is observed during an endoscopic scan, the operating physician may mark the polyp location 504 on the monitoring screen by operating a pre-designated control provided on a handle of the endoscope. In other embodiments, a controller or a voice controller of the endoscope may be used to mark the polyp location. As shown in FIG. 5B the polyp location is marked by a dot 504 on the endoscopic image. In one embodiment, a control on the handle may be manipulated to generate an overlay image, matching the location of the polyp, on the display.

FIG. 5C illustrates an endoscopy tool 506 extending into the body cavity, in accordance with an embodiment of the present specification. As also explained with reference to FIG. 3A, the endoscopy tool 506 comprises a scaled catheter portion 501 and a forceps portion 503. The forceps portion 503 comprises an elongate member 505 coupled with a jaw set 507. A forceps needle having a needle tip 508 protrudes from a distal end of the elongate member 505. Jaw set 507 may be placed in a first closed position and a second open position by operating a medical tool control provided on the proximal end of the medical tool found outside the endoscope handle, in accordance with an embodiment of the present specification. In a first closed position as described above, jaw set 507 closes around the needle tip 508, thereby covering the needle tip 508. In an open position the jaw set 507, which is shown in FIG. 5C, jaw set 507 opens up to expose needle tip 508.

In an embodiment, the endoscopy tool 506 is inserted into the body cavity via a service/working channel of the endoscope (shown in FIG. 1), once the location of the polyp is identified. In various embodiments, when the endoscopy tool is inserted into the body cavity via the service/working channel of the endoscope, the jaw set 507 is in the first closed position thereby protecting the needle tip 508 as well as walls of the service channel. The endoscopy tool 506 extends out of an opening in a tip portion of the endoscope through a service channel opening located therein. In an embodiment, the service/working channel opening is situated on a front panel of the endoscope tip, whereas in another embodiment, the service/working channel opening is situated on a side panel of the endoscope tip.

Once the tool is extended out from the endoscope tip and is positioned in proximity of the polyp 504, the needle tip 508 is uncovered/exposed by operating a pre-designated control causing jaw set 507 to open up and take the second position. Needle tip 508 is placed in closed proximity of the polyp location 504 by observing the same on the monitoring screen (shown in FIG. 1). Once the tool is extended out from the endoscope tip in the first closed position and is placed in closed proximity of the polyp location 504, the scaled catheter portion 501 of the tool 506 extends from the endoscope tip to the polyp 504 showing a plurality of markings 512.

FIG. 5D illustrates an endoscopy tool exiting from an endoscope's tip, in accordance with an embodiment of the present specification. As shown in FIG. 5D, endoscopy tool 506 extends outwards from the endoscope's tip portion 514. FIG. 5D illustrates an open position of jaw set 507. In the open position jaw set 507 lies in a plane perpendicular to the plane of elongate member 505, exposing the needle tip 508 completely, as shown. For measuring the size of a polyp such as polyp 504 (shown in FIGS. 5A, 5B and 5C), needle tip 508 of the tool 506 is placed in proximity to the polyp 504. The scaled portion 501 comprises markings 512 which are placed at predetermined incremental distances along the length of the endoscopy tool 506. The markings 512 and the known length of elongate member 505 and needle tip 508 enable determination of the distance of the polyp 504 from an end 516 of the endoscope's tip portion 514.

FIG. 5E illustrates an endoscopy tool exiting from an endoscope's tip, in accordance with another embodiment of the present specification. As shown in FIG. 5E, endoscopy tool 506 extends outwards from the endoscope's tip portion 514. FIG. 5E illustrates a closed position of jaw set 507. For measuring the size of a polyp such as polyp 504 (shown in FIGS. 5A, 5B and 5C), distal end of jaw set 507 of the tool 506 is placed in proximity to the polyp 504. The scaled portion 501 comprises markings 512 which are placed at predetermined incremental distances along the length of the endoscopy tool 506. In the closed position of the jaw set 507 of medical tool 506, the markings 512 and the known length of elongate member 505 and jaw set 507 enable determination of the distance of the polyp 504 from an end 516 of the endoscope's tip portion 514.

FIG. 5F illustrates a handle of an endoscope comprising a control for operating a scale function, in accordance with an embodiment of the present specification. As shown in FIG. 5E, handle 518 comprises a button 520, which when pressed activates a scale function. Referring to FIGS. 5A, 5B, 5C, 5D and 5E, in an embodiment, an image processor/controller (not shown) coupled with the endoscope executes a scale function which uses the markings 512 of tool 506 visible on the monitoring screen to obtain a distance of the polyp 504 from the end 516 of endoscope tip 514. In an embodiment, the scale function is defined by a plurality programmatic instructions that obtain the markings 516 and convert the readings to a pre-defined scale for obtaining the polyp distance as well as the polyp size. The obtained distance is extrapolated by using a pre-defined marking to scale conversion to determine the size of the polyp 504. One of ordinary skill in the art would appreciate that the marking to scale conversion depends on the amount of separation between the markings and may be generally obtained empirically.

FIG. 5G illustrates an image of the internal walls of a body cavity displaying a polyp location as well as the size of the polyp, in accordance with an embodiment of the present specification. The size 522 of the polyp 504, as determined by using the scale function, is displayed as ‘1 mm’ within the image 502 being observed on the monitoring screen. In various embodiments a size measurement 522 of the observed polyp 504 may be displayed in any pre-defined unit at any position on the monitoring screen. It should further be appreciated that the displayed unit sizing, which may be overlaid on an image, can be determined by obtaining the markings, processing the markings by extrapolating the number and separation between the markings, using a scale conversion to determine a distance scale, and displaying the distance scale in one or more places on the physical display.

FIG. 5H illustrates an image of the internal walls of a body cavity displayed on a monitor screen showing a marker and an endoscopy medical tool placed in proximity to a polyp/abnormality location, in accordance with another embodiment of the present specification. In an embodiment, marker 530 appears on the monitor screen when a scale function of the endoscope is activated. In other embodiments the marker 530 may be displayed on the screen by operating any predefined control provided on the endoscope or one or more devices coupled with the endoscope. In an embodiment, the location of the marker 530 is read by an image processing module coupled with the endoscope.

FIG. 5I illustrates an image of the internal walls of a body cavity displayed on a monitor screen showing a marker marking a first edge of a polyp, in accordance with an embodiment of the present specification. In an embodiment, once marker 530 is displayed on the screen by activating a predefined scale function; the marker is used to mark a first edge 532 of the polyp by pressing a predefined button provided on the endoscope.

FIG. 5J illustrates an image of the internal walls of a body cavity displayed on a monitor screen showing a marker marking a second edge of the polyp, in accordance with an embodiment of the present specification. In an embodiment, after marking the first edge 532, a second opposite edge 534 of the polyp is marked. FIG. 5K illustrates an image of the internal walls of a body cavity displayed on a monitor screen with a size of an observed polyp displayed therein, in accordance with an embodiment of the present specification. The determined size 522 of the polyp 504 is displayed as ‘1 mm’ within the image 502 being observed on the monitoring screen. In an embodiment, once the two edges 532, 534 of the polyp 504 are marked, a size of the polyp is determined by using a predefined method based on the distance between the two marked edges and the number of markings 512 of the endoscopy tool 506 displayed while the needle tip 508 is placed in close proximity to the polyp 504. Having translated a scale on to display and having identified on the display two points representing the polyp size, a size of the polyp can be determined by comparing the distance between the two points to the scale, i.e. comparing the pixel distance from 532 to 534 to the pixel distance for the displayed unit (i.e., “1 mm”) and deriving a corresponding length.

In various embodiments, the scale function comprises a plurality of pre-defined method steps stored as a software module within an image processing module of the controller unit of the endoscope.

In an embodiment, a three dimensional image of the polyp observed in an internal wall of a body cavity being endoscopically scanned may also be obtained by placing a tip of the endoscopy medical tool in proximity to different parts of the polyp. In an embodiment, a three dimensional image of the polyp may be obtained by placing a distal end of the endoscopy medical tool on a proximal end of the polyp, a distal end of the polyp and then across the length of the polyp. Hence, the three dimensional image may be obtained by measuring a longitudinal dimension of the polyp as well as the height of the polyp.

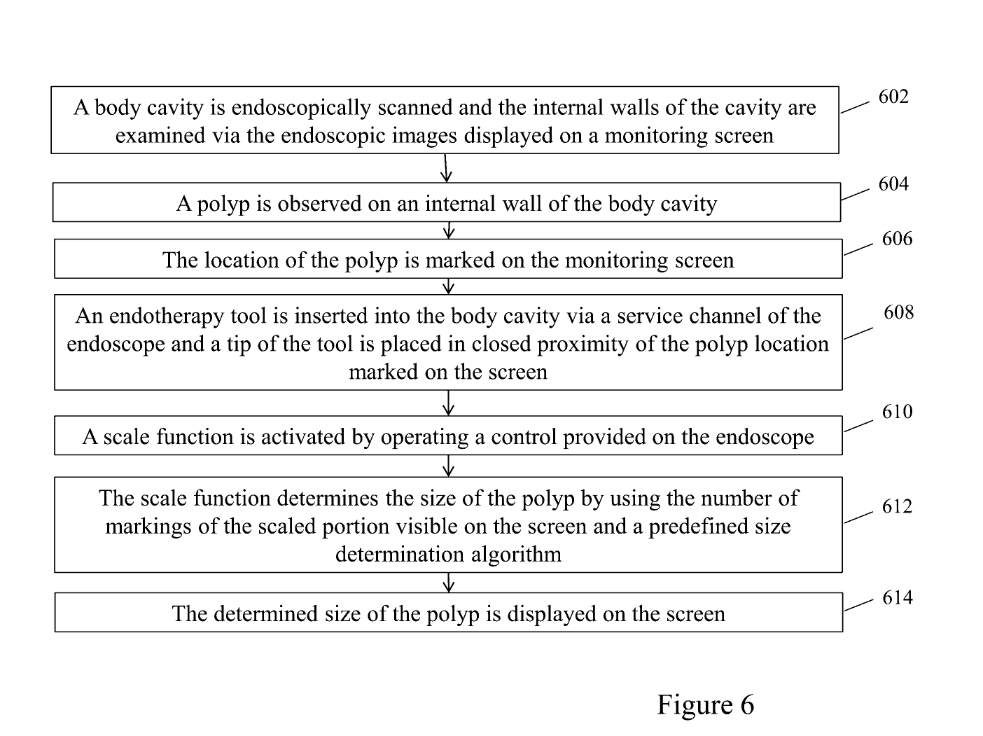

FIG. 6 is a flowchart illustrating the steps for determining the size of a polyp observed within a body cavity being scanned by an endoscope, in accordance with an embodiment of the present specification. At step 602, a body cavity is endoscopically scanned and the internal walls of the cavity are examined via the endoscopic images displayed on a monitoring screen. At step 604 a polyp is observed on an internal wall of the body cavity. At step 606 the location of the polyp is marked on the monitoring screen. At step 608 an endoscopy medical tool is inserted into the body cavity via a service/working channel of the endoscope and a tip of the tool is placed in close proximity of the polyp location marked on the screen. A scaled catheter portion of the tool extends from a distal tip of the endoscope and ends in the tip of the medical tool. The scaled catheter portion comprises a plurality of markings placed at predetermined distances along the length of the tool. At step 610 a scale function is activated by operating a control provided on the endoscope. At step 612 the scale function determines the size of the polyp by using the number of markings of the scaled catheter portion visible on the screen and a predefined size determination algorithm. At step 614 the determined size of the polyp is displayed on the screen.

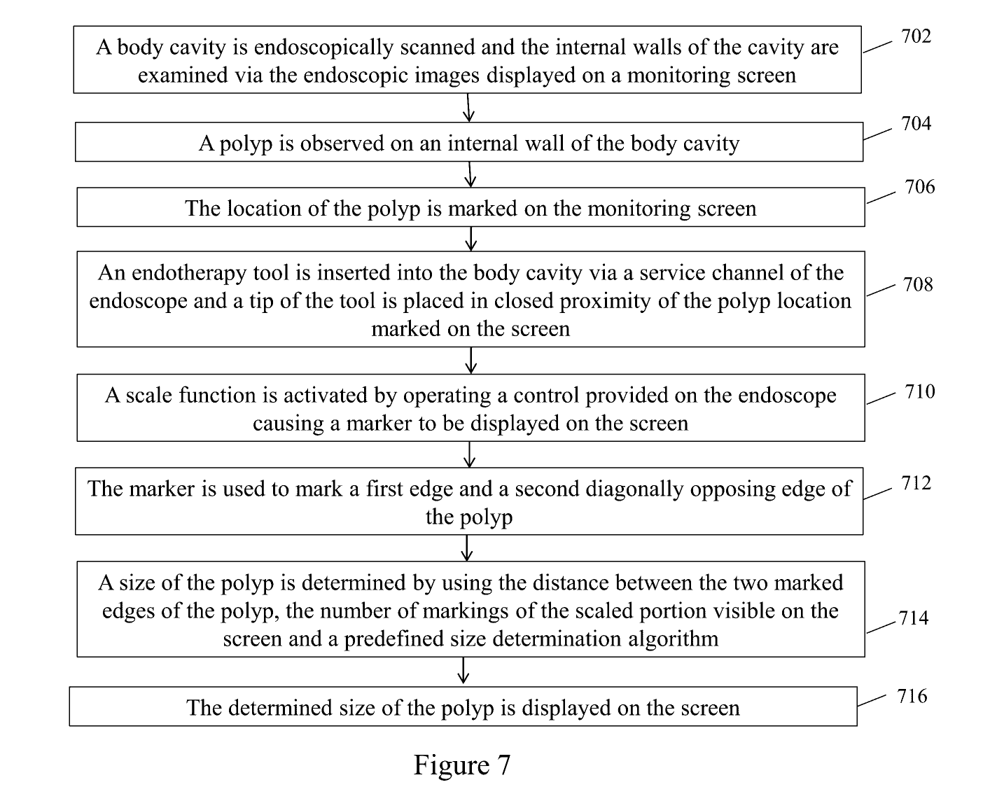

FIG. 7 is a flowchart illustrating the steps for determining the size of a polyp observed within a body cavity being scanned by an endoscope, in accordance with another embodiment of the present specification. At step 702, a body cavity is endoscopically examined and the internal walls of the cavity are examined via the endoscopic images displayed on a monitoring screen. At step 704 a polyp is observed on an internal wall of the body cavity. At step 706 the location of the polyp is marked on the monitoring screen. At step 708 an endoscopy tool is inserted into the body cavity via a service channel of the endoscope and a tip of the tool is placed in closed proximity of the polyp location marked on the screen. A scaled catheter portion of the tool extends from a tip of the endoscope and ends in the tip of the tool. The scaled catheter portion comprises a plurality of markings placed at predetermined distances along the length of the tool. At step 710 a scale function is activated by operating a control provided on the endoscope causing a marker to be displayed on the screen. At step 712 the marker is used to mark a first edge and a second diagonally opposing edge of the polyp. At step 714 a size of the polyp is determined by using the distance between the two marked edges of the polyp, the number of markings of the scaled portion visible on the screen and a predefined size determination algorithm. At step 716 the determined size of the polyp is displayed on the screen.

The above examples are merely illustrative of the many applications of the system of present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. An endoscopy tool for measuring a size of a polyp observed on an internal wall of a body cavity being scanned by an endoscope, wherein the endoscopy tool is adapted to be inserted within the body cavity via a channel of the endoscope, the endoscopy tool comprising:
a scaled catheter portion having a plurality of markings for determining a unit of distance within the body cavity, wherein the scaled catheter portion has a proximal and a distal end;
an operating portion comprising an elongate member coupled with a biopsy tool, wherein the elongate member has a distal end and a proximal end coupled with the distal end of the scaled portion and wherein the biopsy tool is coupled with the distal end of the elongate member; and
a processing unit adapted to determine the unit of distance from said plurality of markings using a scale conversion and to transmit data to a display, wherein said data is indicative of the unit of distance.

2. The endoscopy tool as claimed in claim 1 wherein the biopsy tool comprises at least one of a pair of jaws, a needle, and a net.

3. The endoscopy tool as claimed in claim 1 wherein a distance between any two markings on the scaled catheter portion ranges from one of 0.01 mm to 1 mm.

4. The endoscopy tool as claimed in claim 1 wherein a distance between markings closer to the distal end is smaller as compared to a distance between markings on the scaled catheter portion farther away from the distal end.

5. The endoscopy tool as claimed in claim 1 wherein the elongate member comprises a plurality of markings for determining a distance of the polyp from the distal end of the scaled catheter portion.

6. The endoscopy tool as claimed in claim 1 wherein lengths of the elongate portion and the biopsy tools are pre-defined and wherein the lengths are adapted to be used for determining the distance of the polyp from the tip of the endoscope.

7. The endoscopy tool as claimed in claim 1 having a length ranging between 210 to 240 cm.

8. The endoscopy tool as claimed in claim 1, wherein the endoscopy tool is adapted to exit the endoscope via an opening in a tip portion of the endoscope.

9. The endoscopy tool as claimed in claim 1 wherein the biopsy tool comprises a jaw set having a plurality of flanges, a base end of each of the flanges of the jaw set being coupled with the distal end of the elongate member, the distal end of the elongate member comprising a needle having a tip extending from the distal end of the elongate member in the plane of the elongate member, the tip of the needle being positioned in close proximity of the polyp for determining the distance of the polyp from the tip of the endoscope, wherein the flanges of the jaw set may be placed in a first closed position and a second open position by operating a control; the flanges lying in a plane parallel to the plane of the elongate member in the first position enclosing the needle tip; and the flanges lying in a plane perpendicular to the plane of the elongate member in the second position exposing the needle tip.

10. The endoscopy tool as claimed in claim 1 wherein the processing unit is configured to determine a size of the polyp by using the unit of distance from said plurality of markings and a distance between two edges of the polyp.

11. A method of measuring a size of a polyp observed on an internal wall of a body cavity being scanned by an endoscope by using an endoscopy tool comprising at least a scaled catheter portion having a plurality of markings for determining a unit of distance, the scaled catheter portion having a proximal and a distal end; and an operating portion comprising an elongate member coupled with a biopsy tool, the elongate member having a distal end and a proximal end coupled with the distal end of the scaled portion, the biopsy tool being coupled with the distal end of the elongate member; the method comprising the steps of:
examining internal walls of the body cavity via a displayed endoscopic image;
locating a polyp on an internal wall of the body cavity via the displayed endoscopic image;
marking the location of the polyp on the displayed endoscopic image;
inserting the endoscopy tool into the body cavity via a service channel of the endoscope;
placing a distal tip of the biopsy tool in proximity to the polyp location, the scaled catheter portion of the tool extending from an opening in the tip of the endoscope;
activating a scale function by operating a control provided on the endoscope for determining a size of the polyp by determining a distance of the polyp from a tip of the endoscope and a predefined algorithm; and
displaying the determined size of the polyp in the endoscopic image.

12. The method as claimed in claim 11 wherein the distance of the polyp from the tip of the endoscope is determined by using the plurality of markings of the scaled catheter portion visible in the endoscopic image.

13. The method as claimed in claim 11 wherein the predefined algorithm is a size determination algorithm for measuring the size of the polyp by using the distance of the polyp from the tip of the endoscope.

14. The method as claimed in claim 11 wherein the markings on the scaled catheter portion represent a predefined unit of measurement.

15. A method of measuring a size of a polyp observed on an internal wall of a body cavity being scanned by an endoscope by using an endoscopy tool comprising at least a scaled catheter portion having a plurality of markings for determining a distance of the polyp from a tip of the endoscope, the scaled catheter portion having a proximal and a distal end; and an operating portion comprising an elongate member coupled with a biopsy tool, the elongate member having a distal end and a proximal end coupled with the distal end of the scaled portion, the biopsy tool being coupled with the distal end of the elongate member; the method comprising the steps of:
examining internal walls of the body cavity via a displayed endoscopic image;
locating a polyp on an internal wall of the body cavity via the displayed endoscopic image;
marking the location of the polyp on the displayed endoscopic image;
inserting the endoscopy tool into the body cavity via a service channel of the endoscope;
placing a distal tip of the endoscopy tool in closed proximity of the polyp location, the scaled catheter portion of the tool extending from a tip of the endoscope;
activating a scale function by operating a control provided on the endoscope for marking a first and a second opposing edge of the polyp by a marker displayed in the endoscopic image;
determining the size of the polyp by using the number of markings of the scaled catheter portion visible in the endoscopic image, the distance between the two marked edges of the polyp and a predefined size determination algorithm; and
displaying the determined size of the polyp in the endoscopic image.

16. The method as claimed in claim 15 wherein the biopsy tool is positioned in close proximity of the polyp for determining the distance of the polyp from the tip of the endoscope.

17. The method as claimed in claim 15 wherein the markings on the scaled catheter portion represent a predefined unit of measurement.

18. The method as claimed in claim 15 further comprising rendering a three dimensional image of the polyp by measuring a longitudinal dimension of the polyp and a height of the polyp.

19. The method as claimed in claim 18 wherein the longitudinal dimension and the height of the polyp is measured by placing a distal end of the endoscopy medical tool on a proximal end of the polyp, a distal end of the polyp and across the length of the polyp and using the predefined size determination algorithm.
